(19) United States
(12) Patent Application Publication
(10) Pub. No.: US 2016/0174893 A1
(43) Pub. Date: Jun. 23, 2016

(54) Apparatus and method for nighttime distress event monitoring

(71) Applicant: Vivek LAM, Melville, NY (US)

(72) Inventor: Vivek LAM, Melville, NY (US)

(21) Appl. No.: 14/998,108

(22) Filed: Dec. 23, 2015

Related U.S. Application Data
(60) Provisional application No. 62/096,230, filed on Dec. 23, 2014.

(51) Int. Cl. A61B 5/00

(52) U.S. Cl. CPC A61B 5/4812; A61B 5/746; A61B 5/0004

(57) ABSTRACT

An apparatus and method for nighttime distress event monitoring are provided. The apparatus includes a plurality of sensors configured to measure a sleep state of a user; a processor configured to determine, based upon a combination of information received from the plurality of sensors, whether the user is in a distressed state; a memory configured to store the information obtained from the plurality of sensors; and a communication unit configured to, if the processor determines that the user is in the distressed state, transmit a notification of the distressed state to an external device.

### PRIORITY

This application claims priority under 35 U.S.C. §119(a) to a United States Provisional Application filed in the United States Patent and Trademark Office on Dec. 23, 2014 and assigned Ser. No. 62/096,230, the entire content of which is incorporated herein by reference.

### BACKGROUND

1. Field of the Invention

The present invention generally relates to sleep tracking, and more specifically to an apparatus and method for performing nighttime sleep monitoring.

2. Description of the Related Art

Poor sleep is correlated with mental distress, psychosis, or diseases, such as Alzheimer's and Obstructive Sleep Apnea (OSA). These conditions pose a problem for doctors and therapists, who have limited ability to interact with subjects at home.

Medical crises such as a heart attack or stroke can strike while a person is sleeping. Roommates or family members may not find the person until morning, when the person has passed and the opportunity for essential medical intervention is gone. Furthermore, deaths by suicide peak during the night, possibly brought on by the stress of insomnia, nightmares, or the absence of human contact. Therefore, sleep monitoring may be beneficial during these night time hours, especially for those at risk of either physical or mental health crises.

One current method for performing sleep disorder diagnosis is overnight polysomnography (PSG), which may require a subject to be admitted to a hospital or sleep center. PSG measures limb movement, brain activity using an electroencephalogram (EEG), eye movement using an Electrooculogram (EOG), muscle activity using an Electmmyograms (EMG), skeletal muscle actfvaffun (EMG), and heart rhythm using an electrocardbgram (ECG) during sleep. The markers are recorded concurrently to make correlations obvious. However, the cost of monitoring a person overnight and the scarcity of sleep center beds make extended monitoring via PSG unsustainable. Further, differences between the sleep environment at a sleep center and a normal bedroom make such monitoring unrealistic.

An in-home alternative to PSG is actigraphy, a non-invasive method of monitoring human rest/activity cycles. A small actigraph unit is worn on the wrist or near the hip by the subject to measure gross motor movement. Actigraphs have a number of different ways of accumulating the values from the accelerometer in memory, such as by counting zero crossings (Zero Crossing Mode), measuring the area under the curve for accelerometer signals (Proportional Integral Made), and measuring the time above a certain signal value threshold’ (Time above Threshold). Other in-home alternatives to PSG include Bluetooth devices that use an EEG to measure sleep data.

### SUMMARY OF THE INVENTION

The present invention has been made to address at least the above problems and/or disadvantages and to provide at least the advantages described below.

According to an aspect of the present invention, a sleep monitoring device is provided. The sleep monitoring device includes a plurality of sensors configured to measure a sleep state of a user; a processor configured to determine, based upon a combination of information received from the plurality of sensors, whether the user is in a distressed state; a memory configured to store the information obtained from the plurality of sensors; and a communication unit configured to, if the processor determines that the user is in a distressed state, transmit a notification of the distressed state to an external device.

According to an aspect of the present invent, a method performed by an electronic device for monitoring distress events is provided. The method includes measuring, by a plurality of sensors, a sleep state of a user; storing information obtained from the plurality of sensors; determining, based upon a combination of information received from the plurality of sensors, whether the user is in a distressed state; and transmitting, if the processor determines that the user is in the distressed state, a notification of the distressed state to an external device

### BRIEF DESCRIPTION OF THE DRAWINGS

The above and other aspects, features, and advantages of the present invention will be more apparent from the following detailed description taken in conjunction with the accompanying drawings, in which:

FIG. 1 is a diagram illustrating network environment according to an embodiment of the present invention;

FIG. 2 is a diagram illustrating a sleep monitoring device according to an embodiment of the present invention;

FIG. 3 is a diagram illustrating a sleep alert server according to an embodiment of the present invention;

FIG. 4 is a diagram illustrating a database according to an embodiment of the present invention;

FIG. 5 is a flowchart illustrating a method performed by a sleep monitoring device according to an embodiment of the present invention.

### DETAILED DESCRIPTION OF EMBODIMENTS OF THE PRESENT INVENTION

Hereinafter, embodiments of the present invention will be described in detail with reference to the accompanying drawings. In the description herein, well-known functions and structures which may unnecessarily obscure the subject matter of the present invention may be omitted. The following description includes various specific details to assist in that understanding but these are to be regarded as mere examples. Accordingly, those of ordinary skill in the art will recognize that various changes and modifications of the various embodiments described herein can be made without departing from the scope and spirit of the present invention. Throughout the drawings, like reference numerals may be used to refer to like parts, components, and structures.

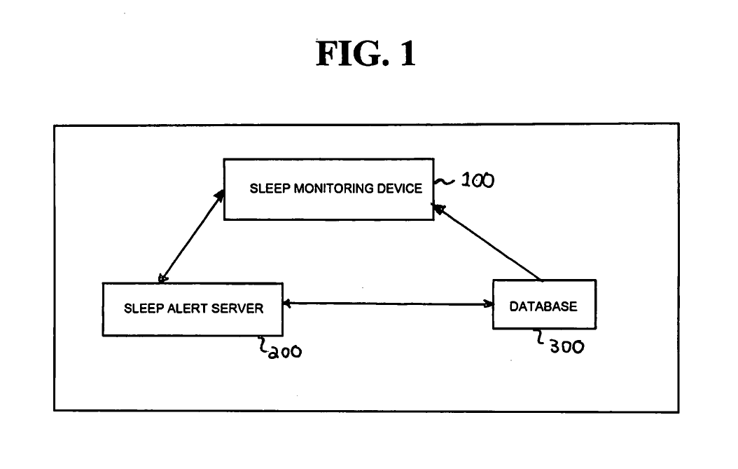

FIG. 1 is a diagram illustrating network environment according to an embodiment of the present invention.

Referring to FIG. 1 of the present application, a network environment includes a sleep monitoring device 100, a sleep alert server 200, and a database 300.

The sleep monitoring device 100 may be embodied as personal electronic device, such as a mobile terminal, a tablet, netbook, portable media player, portable personal computer, a wearable computing device, such as a smartwatch, or other such portable electronic devices that may be located near a user while the user is sleeping. Accordingly, a sleep monitoring device according to embodiments of the present invention is not limited to the components of the present embodiment, and may include additional components of various personal electronic devices in accordance with embodiments of the present invention.

The sleep monitoring device 100 is placed close to a user while a user is sleeping, such as on a mattress or pillow near a face of the user, and sensors included in the sleep monitoring device 100 obtain data corresponding to a user's sleep conditions, such as movement, breathing rate, ambient light, etc.

The sleep monitoring device 100 may regularly send obtained sleep data to the sleep alert server 200 and/or the database 300, and may further send a notification to the sleep alert server 200 if the sleep monitoring device 100 determines that obtained sleep data corresponds to a distress condition, such as, for example, a breathing rate below a predetermined threshold, abrupt movements, cessation of breathing, sleep apnea, or a call for help. Although these distress conditions are provided as examples, other distress conditions may be determined based on information received from sensors in accordance with embodiments of the present invention.

Stress events can be determined based on a combination of information received from a plurality of sensors. For example, the sleep monitoring device 100 may determine that a user has stopped breathing based on a combination of breath rate measurements determined based on measured temperature and movement measurements obtained through accelerometers of the sleep monitoring device 100.

Sleep alert server 200 receives sleep date from the sleep monitoring device 100 and evaluates the received sleep data, either on its own, or in combination with additional sleep data obtained from the database 300 to operate a data collection service and an alert notification service. The additional sleep information may include archived sleep data of the same user and/or sleep data corresponding to other users. This additional data may be used to determine changes in a user's sleep patterns over an extended period of time, and or comparison with other users to determine whether sleep patterns of a user fall within a healthy and/or normal range.

In operating the data collection service, the sleep alert server 200 may receive sleep data from the sleep monitoring device 100 and stores received data in a storage of the sleep alert server 200 and/or the database 300.

In operating the alert notification service, the sleep alert server 200 may determine that received data corresponds to conditions for a notification and/or receive a notification from the database 300, and communicate the notification to the sleep monitoring device 100 (e.g., for lower-priority alerts) via, for example, a Short Message Service (SMS) gateway. In addition to, or as an alternative to alerts sent to the sleep monitoring device 100, the sleep alert server 200 may also send emergency alerts to other recipients designated according to user settings and/or common settings defined for multiple users.

The database 300 stores sleep data from the sleep monitoring device 100 as well as sleep data corresponding to other users and monitoring devices. The database 300 also operates an analytics engine that mines the sleep data and presents the mined data to authorized counselors and health care professionals. The database 300 also performs pattern matching that uses users' sleep patterns and personal history to develop information regarding normal and/or healthy sleep patterns, in order to improve criteria for determining whether sleep data corresponds to a particular event, such as a sleep distress condition, or whether received speech data corresponds to fear or panic of a user. The database 300 may also provide updated criteria for determination of particular events to the sleep monitoring device 100 and/or the sleep alert server 200.

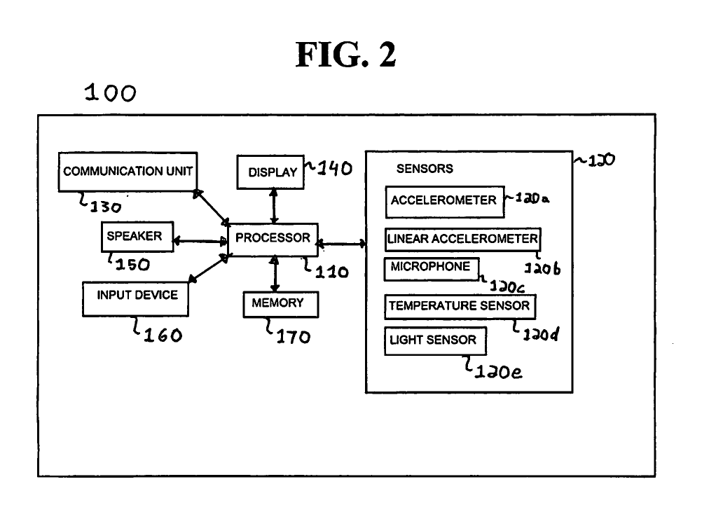

FIG. 2 is a diagram illustrating a sleep monitoring device according to an embodiment of the present invention.

Referring to FIG. 2 of the present application, a sleep monitoring device 100 includes a processor 110, sensors 120, a communication unit 130, a display 140, a speaker 150, an input device 160, and a memory 170.

The processor 110 controls overall operations of the sleep monitoring device 100, including executing a sleep monitoring application stored in memory 170, controlling sensors 120 to obtain sleep data of a user, process the obtained sleep data, and provide information based on the processed sleep data to another device via communication unit 130, and/or provide information based on the processed sleep data to a user via, for example, the display 140, the speaker 150, or any other output device of the sleep monitoring device 100.

The sensors 120 include, for example, an accelerometer sensor 120a, a linear accelerometer sensor 120b, a microphone 120c, a temperature sensor 120d, and a light sensor 120e.

The accelerometer sensor 120a measures acceleration of movement of the sleep monitoring device 100 in three spatial axes (x, y, and z), including gravitational acceleration. The linear accelerometer sensor 120b measures acceleration of movement of the sleep monitoring device 100 three physical axes (x, y, and z), excluding gravitational acceleration. When the sleep monitoring device 100 is placed near a sleeping user, such as, for example, on a same mattress upon which a user is sleeping, the movement of the sleep monitoring device 100 corresponds to movement of the sleeping user, and accordingly, the accelerometer sensor 120a and the linear accelerometer sensor 120b sense acceleration corresponding to the sleeping user's movements.

The accelerometer sensor 120a and the linear accelerometer sensor 120b detect the overall acceleration of the sleep monitoring device 100, including gravitational acceleration. To eliminate the influence of gravitational acceleration on the measurements, the sleep device 100 may use a low pass fitter (LPF) and a high pass filter (HPF). The cascade of these filters rejects the signal noise caused by the force of gravity, retaining only the pure accelerometer data. The LPF isolates the gravitational acceleration, and the HPF removes that component from the accelerometer signal. The filtering is accomplished as follows.

Isolating the gravitational acceleration with the LPF may be performed based upon the following equations:

gravity[0]=alpha*gravity[0]+(1−alpha)*event.values[0] (1)

gravity[1]=alpha*gravity[1]+(I−alpha)*event.values[1] (2)

gravity[2] =alpha*gravity[2]+(1−alpha)*event.values[2] (3)

In this example, a calculated as t1(t+dT), where t is the LPF's time constant and dT is the event delivery rate.

Removal of the gravitational acceleration component with the HPF may be performed based upon the following equations:

linear_acceleration[0]=event.values[0]−gravity[0] (4)

linear acceleration [1]=event.values[0]−gravity[1] (5)

linear acceleration [2]=event.values[0]−gravity[2] (6)

After obtaining the pure acceleration values via the accelerometer sensor 120a and the linear accelerometer sensor 120b, the user's body movement can be detected with precision. Acceleration is then computed from the three components in the X, Y, and Z directions respectively.

A resultant acceleration value may be calculated by, for example, a square root of a sum of the respective squares of the acceleration in each of the X, Y, and Z directions. For the resultant acceleration value, there may be a constraint that the resultant acceleration value must be at least equal to a predetermined value in order for the sleep monitoring device 100 to recognize an acceleration as corresponding to movement of the user. This detected movement may be designated as an “A_value.”

The microphone 120c detects both sound generated by the subject's movement as well as ambient noise, which may be from the signal, in order to more accurately measuring the user's movement. The refined microphone sensed value generated by subject movements after removing the ambient noise may be designated as an “M_value.

The processor 110 may A_value obtained based on measurements from the accelerometer sensor 120a and the linear accelerometer sensor 120b and the M_value obtained based on measurements from the microphone 120c to from a comprehensive indicator of movement of the user, such as according to the following equation:

Final Result (̅A̅_̅v̅a̅l̅u̅e̅+̅M̅₁̅₃̅ v̅a̅l̅u̅e̅)̅=\sqrt{(A_value)²+(M_value)²} (7)

The microphone 100 may also detect a user's voice when the user is awakened. For example, if the user requests help, the sleep monitoring app may recognize the user's request and automatically initiate a communication with the sleep alert server 200 or other service provider, such as an emergency service provider.

The temperature sensor 120d measures temperature at the location of the sleep monitoring device 100. Exhaled air from a user is warmer than inhaled air. Therefore, when the temperature sensor 120d is placed near a user's face, the temperature sensor 120d is able to sense variations in temperature resulting from a user's breathing. For example, the processor 110 may track the rate of a user's breathing, such as in breaths per minute, over the course of a user's sleep cycle.

The light sensor 120e measures the level of ambient light, in order to track information regarding the environment in which the user sleeps. The light sensor 120e may be a dedicated light sensor, or may be tracked by a device/component of the sleep monitoring device 100 with light sensing capabilities, such as a camera.

Although the above-described examples of sensors are contactless sensors, contact-based sensors for measuring heart rate, temperature, etc. for monitoring user conditions may also be used in addition to or in alternative to the above-described sensors in accordance with embodiments of the present invention.

The communication unit 130 includes a device for performing wired and/or wireless communication, and is used by the sleep monitoring device 100 to communicate with local or remote devices, and may communicate via various protocols, such as near field communication, bluetooth, wifi, and protocols for communication via cellular networks. For example, the communication unit 130 may provide raw sensing data obtained by the sensors, or processed sensing data, such as described above. Also, if the processor 120 determines that the processed sleep information indicates a certain condition, such as a sleep distress condition, this information may be communicated to an external device, such as the sleep alert server 200, via an alert. The communication unit 130 may also communicate with other local electronic devices to obtain additional sleep monitoring data from sensors values obtained by those devices.

The display 140 and the speaker 150 are used to provide information to the user visually and aurally, respectively. For example, the display 140 may display statistical information based on the sleep monitoring, provide an interface for adjusting settings of a sleep monitoring application, and may also display alerts corresponding to certain detected sleep conditions, such as a sleep distress condition, or indications of alerts transmitted to other device. The speaker 150 may provide audio information corresponding to the same or different types of information that may be output via the display 140.

Input device 160 allows the user to provide input to the sleep monitoring device 100. The input device 160 may include for example, touch keys and/or physical keys, a touch panel included in a touch screen display or a separate touch panel. The input device 160 may be used by the user to initiate and/or terminate sleep monitoring, as well as adjust settings of a sleep monitoring application.

The memory 170 may include memory integrated into the sleep monitoring device 100 and/or removable memory, such as a Secure Digital (SD) memory card. The memory 170 stores a sleep monitoring application executed by the processor 110 to perform sleep monitoring, as well as setting information for the application, including communication address information for external devices with which the sleep monitoring device 100 may communicate to perform alerts in response to, for example, a sleep distress condition. The memory 170 may also store raw sensor data obtained by the sensors 120 and/or processed sensor data, after sensor data has been processed by the processor 110 while executing a sleep monitoring application.

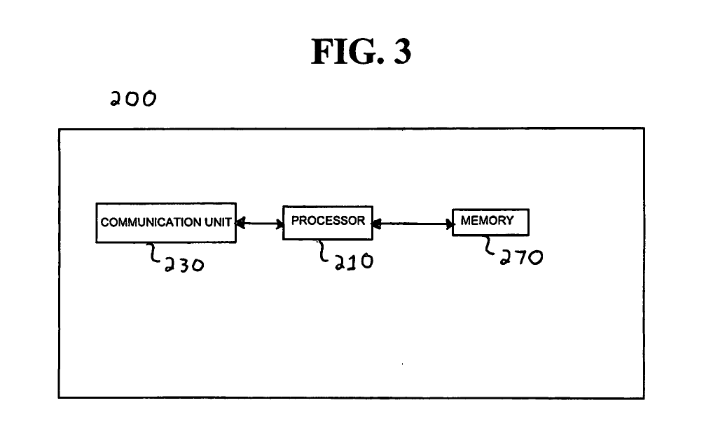

FIG. 3 is a diagram illustrating a sleep alert server according to an embodiment of the present invention.

Referring to FIG. 3 of the present application, a sleep alert server 200 includes a processor 210, a communication unit 230, and a memory 270.

The processor 210 controls overall operations of the sleep alert server 200, including executing a data collection service and an alert notification service according to applications stored in memory 270.

The communication unit 230 obtains sleep data and notifications from the sleep monitoring device 100 and the database 300, as well as provides notifications for specific sleep events, such as a sleep distress condition, to from the sleep monitoring device 100, the database 300, and/or other devices/services, such as contacting an emergency service by phone call or SMS.

The memory 270 stores information including, for example, sleep data of various users/devices, as well as user profile information received from the sleep monitoring device 100, the database 300, and/or other devices.

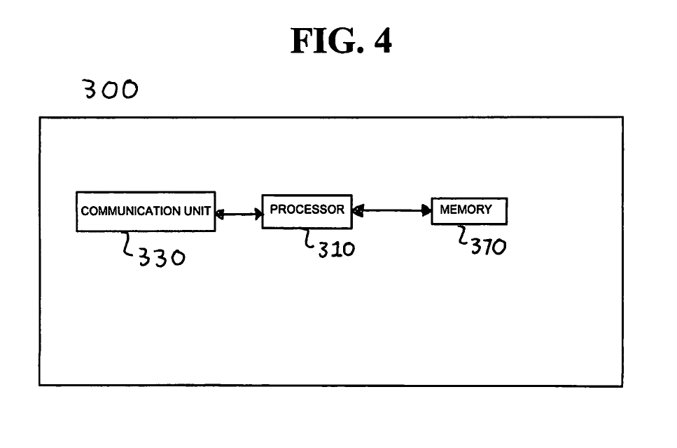

FIG. 4 is a diagram illustrating a database according to an embodiment of the present invention.

Referring to FIG. 4 of the present application, a database 300 includes a processor 310, a communication unit 330, and a memory 370.

The processor 310 controls overall operations of the database 300, including executing an analytics engine and performing pattern matching according to applications stored in memory 370. The processor 310 executes the analytics engine to mine received data according to predetermined criteria, and presents the mined to authorized counselors and health care professionals. The processor 310 performs pattern matching with respect to historical sleep data of a user and/or sleep data of a plurality of users/devices in order to develop more accurate decision criteria for determining whether sleep data satisfies particular conditions, such a crisis event. Aggregated speech data is also used to improve determinations regarding whether sound data received during sleep monitoring corresponds to a voice and/or other sounds indicating fear, panic, and/or other distress or non-distress situations for a user.

The communication unit 330 obtains sleep data and notifications from the sleep monitoring device 100 and the sleep alert server 200, as well as provides notifications for specific sleep events, such as a sleep distress condition, to from the sleep monitoring device 100 and/or the sleep alert server 200. The communication unit 330 may also provide updated criteria for performing methods according to embodiments of the present invention to the sleep monitoring device 100 and/or the sleep alert server 200. For example, updated criteria, such as criteria for determining whether sleep data corresponds to a distress event.

The memory 370 stores information including, for example, sleep data of various users/devices, as well as user profile information received from the sleep monitoring device 100, the sleep alert server 200, and/or other devices.

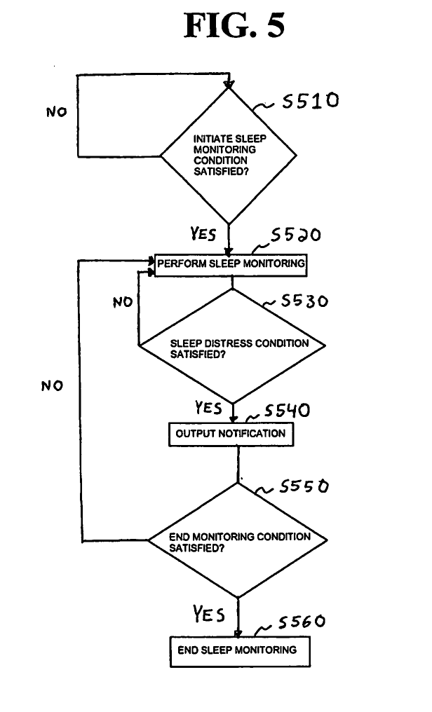

FIG. 5 is a flowchart illustrating a method performed by a sleep monitoring device according to an embodiment of the present invention.

Referring to FIG. 5, at step S510, a sleep monitoring device initiates sleep monitoring. Sleep monitoring may be initiated by a user, such as by manually executing a sleep monitoring application or a sleep monitoring function within the application. The application may also be automatically initiated based on predetermined criteria, such as, for example, one or more of, a breath rate below a predetermined rate, a time of day, sensed ambient light below a predetermined level, ambient temperature, and detected motion of the sleep monitoring device at or below a predetermined level, including for at least a predetermined period of time. Other criteria may also be established based on sensing and/or other capabilities of the sleep monitoring device, in accordance with embodiments of the present invention.

At step S520, the sleep monitoring device uses sensors, including non-contact sensors, such, for example, as a microphone, a light sensor, accelerometers, and a temperature sensor to monitor sleep conditions of a user. During sleep monitoring, raw data from the sensors and/or processed data may be stored in a memory of the sleep monitoring device, and may be transmitted continuously or periodically to a sleep alert server and/or an external database.

At step S530, the sleep monitoring device determines whether obtained sleep data corresponds to a predetermined sleep condition criteria (e.g., a sleep distress condition, such as when a user's breath rate falls below a predetermined rate), or whether a notification corresponding to a predetermined sleep condition is received from a sleep alert server or a database.

At step S540, if the sleep monitoring device determines that sleep data corresponds to a predetermined sleep condition criteria or a notification is received, the sleep monitoring device outputs a notification. The notification may be output by an output device of the sleep monitoring device, such as, for example, a display and/or a speaker. In addition to, or as an alternative, a notification may be output to a sleep alert server and or an external database. If the sleep data does not correspond to a predetermined sleep condition criteria and a notification is not received, sleep monitoring continues at step S530.

At step 550, the sleep monitoring device determines whether a condition for ending sleep monitoring has been satisfied. Sleep monitoring may be terminated by a user, such as by manually executing a sleep monitoring application or a sleep monitoring function within the application. The application may also be automatically terminated based on predetermined criteria, such as, for example, one or more of, a breath rate above a predetermined rate, a time of day, sensed ambient light below a predetermined level, ambient temperature, and detected motion of the sleep monitoring device at or below a predetermined level, including for at least a predetermined period of time. Other criteria may also be established based on sensing and/or other capabilities of the sleep monitoring device, in accordance with embodiments of the present invention.

At step S560, if a condition has ended sleep monitoring sleep device been satisfied, sleep monitoring is terminated. Otherwise, sleep monitoring continues at step S530.

Embodiments of the present invention can be implemented in software, hardware, or a combination thereof. For example, software including instructions for executing a method according to an embodiment of the present invention may be stored, for example, in a storage device such as a Read Only Memory (ROM), a memory such as a Random Access Memory (RAM), a memory chip, or media, such as a Compact Disc (CD), a Digital Versatile Disc (DVD), a magnetic disk.

While the present invention has been particularly shown and described with reference to certain embodiments thereof, various changes in form and detail may be made therein without departing from the spirit and scope of the present invention as defined by the following claims. Accordingly, the scope of the present invention will be defined by the appended claims and equivalents thereof.

What is claimed is:

1. An apparatus for performing distress event monitoring, the apparatus comprising:
a plurality of sensors configured to measure a sleep state of a user;
a processor configured to determine, based upon a combination of information received from the plurality of sensors, whether the user is in a distressed state;
a memory configured to store the information obtained from the plurality of sensors; and
a communication unit configured to, if the processor determines that the user is in the distressed state, transmit a notification of the distressed state to an external device.

2. A method performed by an electronic device for monitoring distress events, the method comprising:
measuring, by a plurality of sensors, a sleep state of a user;
storing information obtained from the plurality of sensors;
determining, based upon a combination of information received from the plurality of sensors, whether the user is in a distressed state; and
transmitting, if the processor determines that the user is in the distressed state, a notification of the distressed state to an external device.
